(19) United States
(12) Patent Application Publication
(10) Pub. No.: US 2024/0398687 A1
(43) Pub. Date: Dec. 5, 2024

(54) HYBRID NAIL COMPOSITION

(71) Applicant: L'ORÉAL, Paris (FR)

(72) Inventors: I-fan HSIEH, Scotch Plains, NJ (US); Ramakrishnan HARIHARAN, Springfield, NJ (US)

(73) Assignee: L’ORÉAL, Paris (FR)

(21) Appl. No.: 18/679,749

(22) Filed: May 31, 2024

Related U.S. Application Data
(60) Provisional application No. 63/469,992, filed on May 31, 2023.

(30) Foreign Application Priority Data
Jul. 26, 2023 (FR) 2308063

(51) Int. Cl. A61K 8/81; A45D 29/00; A61K 8/04; A61K 8/73; A61Q 3/02

(52) U.S. Cl. CPC A61K 8/8147; A45D 29/00; A61K 8/042; A61K 8/731; A61Q 3/02; A61K 2800/594; A61K 2800/81

(57) ABSTRACT

A hybrid cosmetic nail composition may be provided. The composition may include an ultraviolet (UV) gel portion, and a solvent-based portion. The UV gel portion may include a plurality of UV-curable materials and a photoinitiator. The plurality of UV-curable materials may be present in a total amount of at least 19% by weight of the cosmetic nail composition, and the plurality of UV-curable materials may include a difunctional acrylate oligomer. The solvent-based portion may include a plurality of solvents.

### CROSS-REFERENCE TO RELATED APPLICATIONS

The present application claims priority to U.S. Provisional Patent Application No. 63/469,992, filed May 31, 2023, and French Patent application Serial No. FR 2308063, filed Jul. 26, 2023, the contents of which are each incorporated by reference herein in its entirety.

### TECHNICAL FIELD

The present disclosure is drawn to nail compositions, such as UV-curable nail compositions.

### BACKGROUND

Cosmetic nail formulations ideally satisfy numerous consumer needs and desires. For example, it is often desired for nail formulations to have a target color, be able to be used for an extended period of time (e.g., a 2-week wear period, where the color does not substantially change and with high chip resistance), while also being easy to remove. Many features are thought to be technically incompatible. Formulators end up accepting trade-offs to achieve different goals. For example, a formulator may need to use an ingredient which may cause irritation, but causes the formula to last longer on the nails. Such trade-offs are undesirable.

Thus, what is needed is a safer color coat formulation that can still achieve a long use period (e.g., 2 weeks of wear), and can be removed relatively easily.

### BRIEF SUMMARY

A cosmetic nail composition may be provided. The cosmetic nail composition may include an ultraviolet (UV) gel portion and a solvent-based portion. The UV gel portion may include a plurality of UV-curable materials and a photoinitiator (which may be a type I photoinitiator, such as ethyl trimethylbenzoyl phenylphosphinate). The plurality of UV-curable materials may be present in a total amount of at least 19% by weight (such as 19-50%) of the cosmetic nail composition. The solvent-based portion may include a plurality of solvents.

The plurality of UV-curable materials may include a difunctional acrylate oligomer. The difunctional acrylate oligomer may be a urethane monomer, and the plurality of UV-curable materials may include the urethane monomer (such as a urethane acrylate monomer) and a polymerizable (meth)acrylate (such as pyromellitic dimethacrylate). The urethane acrylate monomer may be present in an amount from 8-15% by weight of the composition, and the pyromellitic dimethacrylate may be present in an amount from 2-8% by weight of the composition.

The UV gel portion may be present in a total amount of 19-50% by weight of the cosmetic nail composition. The photoinitiator may be present in a total amount of 3-6% by weight of the cosmetic nail composition.

The solvent-based portion may include at least one film forming agent. The at least one film forming agent may include a cellulose agent (such as nitrocellulose and/or cellulose acetate butyrate). The at least one film forming agent may include an acrylate film former (such as acrylates copolymer, acrylates/isobornyl acrylate copolymer, and/or tricyclodecanedimethanol diacrylate). The at least one film forming agent may include one or more co-film forming agents (such as adipic acid/neopentyl glycol/trimellitic anhydride copolymer). The co-film forming agent may include an epoxy resin (such as a tosylamide epoxy resin). In some embodiments, the acrylate forming agent(s) may be present in a total amount, by weight, that is less than both the total amount of co-film forming agent(s) and the total amount of the cellulose film forming agent(s).

The cosmetic nail composition may include a colorant. The composition may include at least one additional component, the at least one additional component being present in a total amount of no more than 5% by weight of the cosmetic nail composition. The cosmetic nail composition may include a filler, a pH adjusting agent, a UV absorbing agent, or a combination thereof.

In a particular embodiments, (i) the plurality of UV-curable materials may include a first UV-curable material and second UV-curable material, where the first UV-curable material may be present in an amount of 8-15% by weight of the cosmetic nail composition, and the second UV-curable material may be present in an amount of 2-8% by weight of the cosmetic nail composition, (ii) the photoinitiator may be present in an amount of 2-8% by weight of the cosmetic nail composition, (iii) the plurality of solvents may be present in a total amount of 50-70% by weight of the cosmetic nail composition, (iv) the cosmetic nail composition may also include: (a) a plurality of film formers that may be present in a total amount of 5-20% by weight of the cosmetic nail composition, (b) a UV absorbing agent that may be present in a total amount of no more than 1% by weight of the cosmetic nail composition, (c) a pH adjusting agent that may be present in a total amount of no more than 1% by weight of the cosmetic nail composition, (d) a filler that may be present in a total amount of no more than 2% by weight of the cosmetic nail composition, (e) an epoxy resin that may be present in a total amount of no more than 10% by weight of the cosmetic nail composition, and (f) a colorant that may be present in a total amount of no more than 2% by weight of the cosmetic nail composition, where the cosmetic nail composition is substantially free of all other materials.

In various aspects, a method for providing a nail composition with improved wear may be utilized. The method may include applying a composition as disclosed herein to a nail, and curing the composition by exposing the composition to an ultraviolet (UV) light.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

As used herein, the term “at least one” means one or more and thus includes individual components as well as mixtures/combinations.

Other than in the operating examples, or where otherwise indicated, all numbers expressing quantities of ingredients and/or reaction conditions are to be understood as being modified in all instances by the term “about,” meaning within 10% of the indicated number.

As used herein, the term “volatile” means having a flash point of less than about 100° C.

As used herein, the term “non-volatile” means having a flash point of greater than about 100° C.

As used herein, the term “anhydrous” means the compositions contain less than 1% water (by weight). Preferably, anhydrous compositions of the present invention contain less than 0.5% water, and preferably no water.

As used herein, the term “substantially free of (a component)” means that the systems or compositions contain no appreciable amount of the component, for example, no more than about 1% by weight, or no more than about 0.5% by weight, or no more than about 0.3% by weight, such as no more than about 0.1% by weight, based on the weight of the composition.

As used herein, the term “essentially free of (a component)” means that the systems or compositions contain no appreciable amount of the component, for example, no more than about 0.1% by weight, or no more than about 0.01% by weight, based on the weight of the composition.

As used herein, the term “free” or “completely free of (a component)” as defined herein means that the systems or compositions do not contain the component in any measurable degree by standard means.

The compositions and methods of the present invention can “comprise,” “consist of” or “consist essentially of” the essential elements and limitations of the invention described herein, as well as any additional or optional ingredients, components, or limitations described herein or otherwise useful. For purposes of the compositions and methods of the present invention where the invention “consists essentially of” the identified ingredients and/or process steps, the “basic and novel property” of such compositions and/or methods is “a UV-curable nail composition with wear performance and chip resistance comparable to conventional solvent-based nail formulations, that is safer than conventional UV gel formulations.”

A cosmetic nail composition may be provided.

The disclosed compositions can be considered as combination of parts of a UV gel formulation with parts of a solvent-based formulations. Specifically, the cosmetic nail composition may include an UV gel portion and a solvent-based portion. The composition is preferably substantially free, essentially free, or free of water. In a preferred embodiment, the composition is an anhydrous composition.

### UV Gel Portion.

The UV gel portion may include a plurality of UV-curable materials and a photoinitiator. Preferably the UV gel portion consists of the UV-curable materials and the photoinitiator.

The UV gel portion may be present in a total amount of at least 19% by weight of the cosmetic nail composition. In some embodiments, the UV gel portion may be present in a total amount of 19-50% by weight of the cosmetic nail composition. In some embodiments, the UV gel portion may be present in a total amount of 19-34% by weight of the cosmetic nail composition. In some embodiments, the UV gel portion may be present in a total amount of 15-20% by weight of the cosmetic nail composition. In some embodiments, the UV gel portion may be present in a total amount of up to 50% by weight of the cosmetic nail composition. In some embodiments, the UV gel portion may be present in a total amount of up to 45% by weight of the cosmetic nail composition. In some embodiments, the UV gel portion may be present in a total amount of up to 40% by weight of the cosmetic nail composition. In some embodiments, the UV gel portion may be present in a total amount of up to 35% by weight of the cosmetic nail composition. In some embodiments, the UV gel portion may be present in a total amount of up to 34% by weight of the cosmetic nail composition. In some embodiments, the UV gel portion may be present in a total amount of up to 30% by weight of the cosmetic nail composition. In some embodiments, the UV gel portion may be present in a total amount of up to 25% by weight of the cosmetic nail composition. In some embodiments, the UV gel portion may be present in a total amount of up to 20% by weight of the cosmetic nail composition. In some embodiments, the UV gel portion may be present in a total amount of at least 15% by weight of the cosmetic nail composition. In some embodiments, the UV gel portion may be present in a total amount of at least 16% by weight of the cosmetic nail composition. In some embodiments, the UV gel portion may be present in a total amount of at least 17% by weight of the cosmetic nail composition. In some embodiments, the UV gel portion may be present in a total amount of at least 18% by weight of the cosmetic nail composition. In some embodiments, the UV gel portion may be present in a total amount of at least 19% by weight of the cosmetic nail composition. In some embodiments, the UV gel portion may be present in a total amount of at least 20% by weight of the cosmetic nail composition.

### UV-Curable Materials.

The plurality of UV-curable materials may include a difunctional acrylate oligomer.

The difunctional acrylate oligomer may include a urethane monomer. The urethane monomer may be a urethane acrylate monomer. Examples of suitable urethane oligomers include reactive multifunctional oligomers such as polyether urethane acrylates, allyl functional urethane, polybutadiene urethane acrylates, polyethyelene-polybutylene urethane acrylates, and aliphatic or aromatic urethane acrylates. One preferred embodiment utilized a urethane acrylates monomer.

The difunctional acylate oligomer may be present in a total amount of 5-25%. In some embodiments, the difunctional acylate oligomer may be present in a total amount of 8-20%. In some embodiments, the difunctional acylate oligomer may be present in a total amount of 10-15%.

The plurality of UV-curable materials may also include a polymerizable (meth)acrylate. In an embodiment, the term “(meth)acrylate” refers to moieties having an acrylate (CH₂═CHCOO—) or a methacrylate (CH₂═CMeCOO—) group. The polymerizable (meth)acrylate may be a (meth)acrylic ester, which includes esters of acrylic and methacrylic acid.

Non-limiting examples of polymerizable monofunctional (meth)acrylates include methyl (meth)acrylate, ethyl (meth)acrylate, hydroxypropyl (meth)acrylate, butyl (meth)acrylates, hydroxy ethyl (meth)acrylates, butoxyethyl (meth)acrylate, diethylaminoethyl (meth)acrylate, 2-ethylhexyl (meth)acrylate, ethoxyethyl (meth)acrylate, t-butyl aminoethyl (meth)acrylate, methoxyethylene glycol (meth)acrylate, phosphoethyl(meth)acrylate, methoxy propyl (meth)acrylate, methoxy polyethylene glycol(meth)acrylate, phenoxyethylene glycol (meth)acrylate, phenoxypolyethylene glycol (meth)acrylate, 2-hydroxy-3-phenoxypropyl (meth)acrylate, 2-(meth)acryloxyethylsuccinic acid, 2-(meth)acryloylethylphthalic acid, 2-(meth)acryloyloxypropylphthalic acid, stearyl (meth)acrylate, isobornyl (meth)acrylate, 3-chloro-2-hydroxypropyl (meth)acrylates, tetrahydrofurfuryl (meth)acrylate, methacryloyloxyethyl trimellitic anhydride, (meth)acrylamides and allyl monomers.

Not limiting examples of polymerizable difunctional (meth)acryloyl esters include 1,4 butane diol di(meth)acrylate, 1,6 hexanediol di(meth)acrylate, alkoxylated hexane diol di(meth)acrylate, 1,9 nonanediol di(meth)acrylate, 1,10 decanediol di(meth)acrylate, neopentyl glycol di(meth)acrylate, alkoxylated neopentyl glycol di(meth)acrylate, 2-methyl-1,8-octane diol di(meth)acrylate, cyclohexane dimethanol di(meth)acrylate, glycerin di(meth)acrylate, ethylene glycol di(meth)acrylate, triethyleneglycol di(meth)acrylate, polyethylene glycol di(meth)acrylate, propylene glycol di(meth)acrylate, polypropylene glycol di(meth)acrylate, ethoxylated propylene glycol di(meth)acrylate, ethoxylated polypropylene glycol di(meth)acrylate, polyethoxypropoxy di(meth)acrylate, ethoxylated bisphenol A di(meth)acrylate, propoxylated bisphenol A di(meth)acrylate, propoxylated ethoxylated bisphenol A di(meth)acrylate, bisphenol A glycidyl methacrylate, tricyclodecanedimethanol di(meth)acrylate, glycerin di(meth)acrylate, ethoxylated glycerin di(meth)acrylate, bis acrylamides, bis allyl ethers and allyl(meth)acrylates.

Non-limiting examples of polymerizable tri- and/or higher (meth)acrylates include trimethylol propane tri(meth)acrylate, ethoxylated glycerin tri(meth)acrylate, ethoxylated trimethylolpropane tri(meth)acrylate, ditrimethylol propane tetra(meth)acrylate, pentaerythritol tri(meth)acrylate, pentaerythritol tetra(meth)acrylate, propoxylated pentaerythritol tetra(meth)acrylate, ethoxylated pentaerythritol tetra(meth)acrylate, dipentaerythritol hexa(meth)acrylate, dipentaerythritol penta(meth)acrylate and ethoxylated isocyanuric acid tri(meth)acrylates.

Non-limiting examples of polymerizable (meth)acrylates also includes monomers containing acid groups, such as (meth)acrylic acid, bis(glyceryl dimethacrylate) pyromellitate, pyromellitic dimethacrylate, methacryloyloxyethyl phthalate, methacryloyloxyethyl maleate, 2 hydroxyethyl methacrylate/succinate, 1,3 glyceryl dimethacrylate maleate adduct, and 1,3 glyceryl dimethacrylate succinate adduct.

In a preferred embodiment, pyromellitic dimethacrylate is utilized.

In some embodiments, the polymerizable (meth)acrylate may be present in a total amount of 3-15% by weight of the cosmetic nail composition. In some embodiments, the polymerizable (meth)acrylate may be present in a total amount of 4-10% by weight of the cosmetic nail composition. In some embodiments, the ratio, by weight, of difunctional acylate oligomer to polymerizable (meth)acrylate is about 2:1.

### Photoinitiator.

The photoinitiator may be a type I photoinitiator. Non-limiting examples of type I photoinitiators include diphenyl-(2,4,6-trimethylbenzoyl)-phosphine oxide, ethyl(2,4,6-trimethylbenzoyl)phenyl phosphinate, 2-benzyl-2-dimethylamino-1-(4-morpholinophenyl)-butan-1-one, 2-Dimethylamino-2-(4-methyl-benzyl)-1-(4-morpholin-4-yl-phenyl)-butan-1-one and 2-methyl-1-[4(methoxythio)-phenyl]-2-morpholinopropan-2-one. Combinations of type I photoinitiators may also be used. A preferred photoinitiator is ethyl trimethylbenzoyl phenylphosphinate.

The photoinitiator may be present in a total amount of 3-6% by weight of the cosmetic nail composition.

### Solvent-Based Portion.

The solvent-based portion may include a plurality of solvents. The solvent-based portion may include one or more film formers. The solvent-based portion may include a colorant. The solvent-based portion may include one or more additional components, such as a UV absorbing agent, a pH adjuster, and/or a filler.

The solvent-based portion may be present in a total amount of 65%, 70%, or 75% up to 80%, 83%, or 85% by weight of the composition. In some embodiments, the solvent-based portion may be present in a total amount of 65% to 85% by weight of the composition. In some embodiments, the solvent-based portion may be present in a total amount of 75% to 83% by weight of the composition.

### Solvents.

The solvent may be a part of an acetate-rich solvent system. “By acetate-rich solvent system,” it is meant that the proportion of acetate compounds in the solvent system is at least about 30% by weight, such as at least about 50% by weight, such as at least about 70% by weight, such as at least about 80% by weight, such as at least about 85% by weight, where these percentages are relative to all solvents in the composition.

The plurality of solvents may be present in a total amount of at least 50%, at least 55%, or at least 60% by weight of the cosmetic nail composition.

Suitable acetate compounds include, for example, C1-C4 alkyl acetates such as ethyl acetate, propyl acetate, amyl acetate, and butyl acetate.

The solvents used may have a molecular weight less than or equal to 200. One or more of the solvents (and preferably all of the solvents) may have a boiling point between 55° C. and 250° C. In some embodiments, one or more of the solvents may have a boiling point between 120° C. and 250° C.

In some embodiments, the composition may include a plurality of acetate compounds. In some embodiments, the plurality of acetate compounds may be present in a total amount of at least 50% by weight of the composition. In one embodiment, two acetate compounds are each present in an amount of at least 20% by weight of the composition, and any other acetate compound is present in a total amount less than 1% by weight of the composition.

The plurality of solvents may include non-acetate compounds that assist in dissolving other components in the system, such as, e.g., nitrocellulose and/or other film formers. This may include C2-C5 monoalcohols such as isopropanol or ethanol. C2-C5 monoalcohols may be present in a total amount no more than 10%, no more than 8%, no more than 6%, or no more than 4% by weight of the composition.

The plurality of solvents may include a volatile oil. As used herein, the term “oil” means any fatty substance in liquid form at room temperature (25° C.) and at atmospheric pressure (1.013.10 5 Pa). As used herein, the term “volatile oil” refers to an oil having in particular a non-zero vapor pressure, at ambient temperature and atmospheric pressure, in particular having a vapor pressure ranging from 2.66 Pa to 40,000 Pa), in particular ranging from 2.66 Pa to 13,000 Pa, and more particularly ranging from 2.66 Pa to 1,300 Pa.

The volatile oil may be hydrocarbon-based and may be non-polar. Non-limiting examples of volatile non-polar hydrocarbon oils include oils having from 8 to 16 carbon atoms, such as C8-C16 branched alkanes, such as isododecane, isodecane, and/or isohexadecane. In one embodiment, the volatile oil is isododecane.

The volatile oil, if present, may be present in a total amount of no more than 5%, no more than 4%, no more than 3%, no more than 2%, or no more than 1.5% by weight of the composition.

The plurality of solvents may include at least one benzoic acid derivative, citric acid derivative, and/or isobutyrate derivative. In one embodiment, at least one benzoic acid derivative, at least one citric acid derivative, and at least one isobutyrate derivative are present.

In certain embodiments, the benzoic acid derivative may be a dibenzoate ester, including but not limited to diethylene glycol dibenzoate, dipropylene glycol dibenzoate, and 1,2-propylene glycol dibenzoate.

In certain embodiments, the citric acid derivative may be an optionally hydroxylated triester of a C2-C8 tricarboxylic acid and of a C2-C8 alcohol, such as citric acid esters, such as trioctyl citrate, triethyl citrate, acetyl tributyl citrate, tributyl citrate or acetyl tributyl citrate.

In certain embodiments, the isobutyrate derivative may be a C1-C6 carboxylic acid ester of sucrose such as sucrose acetate isobutyrate.

In certain embodiments, the at least one benzoic acid derivative, citric acid derivative, and/or isobutyrate derivative may include dipropylene glycol dibenzoate, tributyl citrate, acetyl tributyl citrate, and sucrose acetate isobutyrate.

The at least one benzoic acid derivative, citric acid derivative, and/or isobutyrate derivative may be present in a total amount ranging from about 0.5%, 2% or 3% to about 3%, 4%, 5%, or 8% by weight of the composition. In one embodiment, the total amount is 3%-4% by weight of the composition.

### Film Forming Agents.

The solvent-based portion may include at least one film forming agent. The at least one film forming agent may be present in a total amount of 5%-15% by weight of the composition. In some embodiments, the film forming agent(s) may be present in a total amount of less than (or alternatively no more than) 10% by weight of the composition, such as 6%-10% by weight of the composition.

The at least one film forming agent may include a cellulose film forming agent, an acrylate film forming agent, and/or a co-film forming agent.

Non-limiting examples of the cellulose film forming agent includes nitrocellulose, ethylcellulose, cellulose acetate, cellulose acetate butyrate, cellulose acetate propionate, or mixtures thereof.

In some embodiments, a plurality of cellulose film forming agents are present. In some embodiments, a single cellulose film forming agent is present. The cellulose film forming agent(s) may be present in a total amount of 6%-10% by weight of the composition, such as 7%-9%.

Non-limiting examples of acrylate film forming agents include acrylates copolymer, acrylates/isobornyl acrylate copolymer, acrylic acid/isobutyl acrylate/isobornyl acrylate copolymer, acrylates/stearyl methacrylate copolymer, acrylate/trimethyl polysiloxymethacrylate, acrylates/dimethicone copolymer, octylacrylamide/acrylates/butylaminoethyl methacrylate copolymer, VA/butyl maleate/isobornyl acrylate copolymer, acrylates/t-butylacrylamide copolymer, and acrylates/dimethylaminoethyl methacrylate copolymer. In one embodiment, the acrylate polymer includes acrylates copolymer and acrylates/isobornyl acrylate copolymer.

The acrylate film forming agent(s) may be present in a total amount of 0.5%, 1%, or 1.5% up to 2%, 2.5%, or 3% by weight of the composition. In one embodiment, a plurality of acrylate film forming agents are present, where the first acrylate film forming agent being present in an amount of at least 1.5% by weight of the composition, and the composition is substantially free or essentially free of all other acrylate film forming agents (such as all other acrylate film forming agents being present in a total amount of less than 0.1% by weight of the composition)

Non-limiting examples of co-film forming agents include phthalic anhydride/glycerin/glycidyl decanoate copolymer, adipic acid/diethylene glycol/glycerin crosspolymer, adipic acid/neopentyl glycol/trimellitic anhydride copolymer, tosylamide/epoxy resin, and hydrogenated acetophenone/oxymethylene copolymer.

In some embodiments, the co-film forming agents may include an epoxy resin or a polyester resin.

The co-film forming agent(s) may be present in a total amount of up to 8%, 9%, or 10% by weight of the composition. In some embodiments, the composition may include a plurality of co-film forming agents, including (i) an epoxy resin (such as tosylamide/epoxy resin) in an amount of 5%-10% by weight of the composition, and (ii) one or more additional co-film forming agents, the additional co-film forming agents present in a total amount of 0.1%-0.5% by weight of the composition.

### Colorants.

The composition may include one or more colorants. Any colorant typically found in nail compositions can be used. Non-limiting examples of colorants include lipophilic dyes, pigments and pearlescent agents, and their mixtures.

Non-limiting examples of fat-soluble dyes are, for example, Sudan red, DC Red 17, DC Green 6, beta-carotene, soybean oil, Sudan brown, DC Yellow 11, DC Violet 2, DC Orange 5 and quinoline yellow.

Pigments may be, e.g., white or colored, inorganic and/or organic and coated or uncoated. Mention may be made, for example, of inorganic pigments such as titanium dioxide, optionally surface treated, zirconium or cerium oxides and iron or chromium oxides, manganese violet, ultramarine blue, chromium hydrate and ferric blue. Mention may also be made, among organic pigments, of carbon black, pigments of D & C type and lakes based on cochineal carmine or on barium, strontium, calcium or aluminum, such as D&C Red No. 10, 11, 12, and 13, D&C Red No. 7, D&C Red No. 5 and 6, and D&D Red No. 34, as well as lakes such as D&C Yellow Lake No. 5 and D&C Red Lake No. 2.

In some embodiments, pearlescent pigments can be chosen from, for example, white pearlescent pigments, such as mica covered with titanium oxide or with bismuth oxychloride, colored pearlescent pigments, such as titanium oxide-coated mica with iron oxides, titanium oxide-coated mica with in particular ferric blue or chromium oxide, or titanium oxide-coated mica with an organic pigment of the abovementioned type, and pearlescent pigments based on bismuth oxychloride.

The colorant may be present in the nail composition in a total amount of from 0.01% to 10% by weight, preferably from 0.1% to 5% by weight, and more preferably from 0.5% to 2% by weight of the composition. In some embodiments, the colorant may be present in a total amount of less than 1% by weight of the composition. In some embodiments, the colorant may be present in a total amount of less than 20% by weight of the composition. In some embodiments, the colorant may be present in a total amount of less than 15% by weight of the composition. In some embodiments, the colorant may be present in a total amount of less than 10% by weight of the composition. In some embodiments, the colorant may be present in a total amount of less than 5% by weight of the composition. In some embodiments, the colorant may be present in a total amount of at least 0.01% by weight of the composition. In some embodiments, the colorant may be present in a total amount of at least 1% by weight of the composition. In some embodiments, the colorant may be present in a total amount of at least 5% by weight of the composition.

### Additional Components.

The cosmetic nail composition may include one or more additional components. In one embodiment, the cosmetic nail composition may be substantially free of any additional components (i.e., the composition may include the UV gel portion, the solvents, the film formers, and the colorants, and may be substantially free of any other component).

In some embodiments, the additional component(s) may be present in a total amount of no more than 5% by weight of the cosmetic nail composition. The additional components may include a filler, a pH adjusting agent, a UV absorbing agent, or a combination thereof.

### Fillers.

Non-limiting examples of filler agents (or fillers) include ethyl methacrylate/glycol dimethacrylate crosspolymer, vinyl dimethicone/methicone silsesquioxane crosspolymer, methyl methacrylate crosspolymer, nylon-12, polyamides, polyethylene, talc, titanium dioxide, silica, aluminum starch octenylsuccinate, clays (such as hectorite clays including stearalkonium hectorite), silicas, polymethylsilsesquioxane, and mixtures thereof.

In some embodiments, a plurality of filler agents are present. In some embodiments, only a single filler agent is utilized. In some embodiments, filler agent(s) are present in a total amount of no more than 2% by weight of the composition. In some embodiments, filler agent(s) are present in a total amount of no more than 1.5% by weight of the composition. In some embodiments, filler agent(s) are present in a total amount of no more than 1% by weight of the composition.

### UV Absorbing Agents.

The UV absorbing agent may be any appropriate UV absorbing agent or filter, such as one or more hydrophobic or water-insoluble organic UV filter(s). Non-limiting examples of such filters include the following:
Anthranilic compounds: Menthyl anthranilate.
Dibenzoylmethane compounds: Butyl methoxydibenzoylmethane, and isopropyl dibenzoylmethane.
Cinnamic compounds: Ethylhexyl methoxycinnamate, isopropyl methoxycinnamate; isopropoxy methoxycinnamate; isoamyl methoxycinnamate, cinoxate (2-ethoxyethyl-4-methoxy cinnamate); DEA methoxycinnamate; diisopropyl methylcinnamate; and glyceryl ethylhexanoate dimethoxycinnamate.
Salicylic compounds: Homosalate (homomenthyl salicylate), ethylhexyl salicylate, glycol salicylate; butyloctyl salicylate; phenyl salicylate; dipropyleneglycol salicylate, and TEA salicylate.
Camphor compounds, in particular, benzylidenecamphor derivatives: 3-benzylidene camphor, 4-methylbenzylidene camphor, benzylidene camphor sulfonic acid, camphor benzalkonium methosulfate, and polyacrylamidomethyl benzylidene camphor.
Benzophenone compounds: Benzophenone-1 (2,4-dihydroxybenzophenone), benzophenone-2 (Tetrahydroxybenzophenone), Benzophenone-3 (2-hydroxy-4-methoxybenzophenone) or oxybenzone, benzophenone-4 (hydroxymethoxy benzophenone sulfonic acid), benzophenone-5 (Sodium hydroxymethoxy benzophenone Sulfonate), benzophenone-6 (dihydroxy dimethoxy benzophenone), benzophenone-8, benzophenone-9 (Disodium dihydroxy dimethoxy benzophenonedisulfonate), benzophenone-12, and n-hexyl 2-(4-diethylamino-2-hydroxybenzoyl)benzoate.
β,β-Diphenylacrylate compounds: Octocrylene, and Etocrylene.
Triazine compounds: Diethylhexyl butamido triazone, 2,4,6-tris(dineopentyl 4′-aminobenzalmalonate)-s-triazine, bis-ethylhexyloxyphenol methoxyphenyl triazine, and ethylhexyl triazone.
Benzotriazole compounds, in particular, phenylbenzothiazole derivatives: −(2H-benzotriazole-2-yl)-6-dodecyl-4-methylphenol, branched and linear; and those described in U.S. Pat. No. 5,240,975.
Benzalmalonate compounds: Dineopentyl 4′-methoxybenzalmalonate, and polyorganosiloxane comprising benzalmalonate functional groups, such as polysilicone-15.
Benzimidazole compounds, in particular, phenylbenzimidazole derivatives.
Imidazoline compounds: Ethylhexyl dimethoxybenzylidene dioxoimidazoline propionate.
Bis-benzoxazolyl compounds: The derivatives as described in EP-669,323 and U.S. Pat. No. 2,463,264.
Para-aminobenzoic acid compounds: PABA (p-aminobenzoic acid), ethyl PABA, Ethyl dihydroxypropyl PABA, pentyl dimethyl PABA, ethylhexyl dimethyl PABA, glyceryl PABA, and PEG-25 PABA.
Methylene bis-(hydroxyphenylbenzotriazole) compounds, such as 2,2′-methylenebis[6-(2H-benzotriazol-2-yl)-4-methyl-phenol] in the solid form, 2,2′-methylenebis[6-(2H-benzotriazol-2-yl)-4-(1,1,3,3-tetramethylbutyl)phenol] in the micronized form in an aqueous dispersion, and the derivatives as described in U.S. Pat. Nos. 5,237,071, 5,166,355, GB-2,303,549, DE-197,26,184 and EP-893,119, and Drometrizole trisiloxane as represented below.

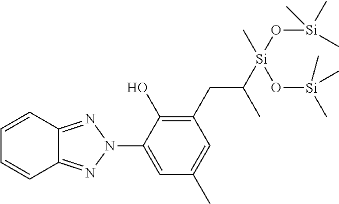 [SMILES: Cc1cc(CC(C)[Si](C)(O[Si](C)(C)C)O[Si](C)(C)C)c(O)c(-n2nc3ccccc3n2)c1]

Benzoxazole compounds: 2,4-bis[5-1(dimethylpropyl)benzoxazol-2-yl-(4-phenyl)imino]-6-(2-ethylhexyl)imino-1,3,5-triazine.

In some embodiments, a plurality of UV absorbing agents are present. In some embodiments, only a single UV absorbing agent is utilized. In some embodiments, UV absorbing agent(s) are present in a total amount of no more than 1% by weight of the composition. In some embodiments, UV absorbing agent(s) are present in a total amount of no more than 0.5% by weight of the composition. In some embodiments, UV absorbing agent(s) are present in a total amount of no more than 0.1% by weight of the composition.

### pH Adjusters.

The composition may also include acid and/or alkali pH adjusters, which are well known in the art. Such pH adjusters include, but are not limited to, sodium metasilicate, silicate compounds, citric acid, ascorbic acid, and carbonate compounds.

In some embodiments, pH adjuster(s) are present in a total amount of no more than 1% by weight of the composition. In some embodiments, pH adjuster(s) are present in a total amount of no more than 0.5% by weight of the composition. In some embodiments, pH adjuster(s) are present in a total amount of no more than 0.1% by weight of the composition.

In some embodiments, the composition may include the UV gel portion, the solvent(s), the film former(s), the colorant(s), filler(s), UV absorbing agent(s), and pH adjuster(s), and may be substantially free, essentially free, or free of any other component.

In various aspects, a method for providing a nail composition with improved wear may be utilized. The method may include applying a composition as disclosed herein to a nail, and curing the composition by exposing the composition to an ultraviolet (UV) light. After wearing (e.g., for an extended period of time), the composition can then be removed from the nail, with less damage to the nail as compared to conventional long-wear UV gel nail formulations.

### EXAMPLES

### Example 1

One embodiment of a nail formulation can be seen in Table 1, below.

| Material | Wt % |
|---|---|
| UV Gel Portion | 19-30% |
| UV Curable Materials | 15-23% |
| Difunctional acrylate oligomer | 8-15% |
| Polymerizable (meth)acrylate | 2-8% |
| Photoinitiator | 2-8% |
| Solvent-Based Portion | 70-81% |
| Solvents | 50-70% |
| Acetates (Ethyl, Propyl, Butyl) | 45-60% |
| C2-C5 Monoalcohols (Ethanol, Isopropanol) | 3-5% |
| C8-C16 Volatile Hydrocarbon | 1-2% |
| Benzoic acid derivative, citric acid derivative, and/or | 3-5% |
| isobutyrate derivative | |
| Film Formers | 5-20% |
| Celluloses (Nitrocellulose) | 3-10% |
| Acrylates | 1-2% |
| Co-Film Former (Epoxy) | 3-10% |
| Co-Film Former (Other) | <1% |
| Colorant | <2% |
| Additional Components (pH Adjusters, Fillers, UV Absorbing | <2% |
| Agents) | |

### Example 2

Three formulations were made, similar to the formula in Example 1, where the percentage of UV curable materials was allowed to vary, to be QS'd with acetates, but the formulations were otherwise identical to each other. The three formulations had 8%, 11%, and 19% UV curable materials.

To evaluate the samples, each test formula was painted onto a nail spoon, by applying 2 coats of each formula onto a target nail spoon. Each nail spoon was then cured under an LED UV light for 30 sec. After curing with the UV light, each nail spoon was aged at room temperature for 24 hours before a removal test.

The removal test was performed as follows. A cotton pad was saturated with 1 ml of acetone. The nail spoon was then rubbed in a single direction using the cotton pad, repeatedly, until the color coat is removed or the acetone in cotton pad is gone. The number of strokes (or passes) required to remove the color is counted. A higher number of strokes in this removability test indicates the formulation is more difficult to remove, and the crosslinking density is higher. The results are shown in Table 2, below.

| Nail Spoon # | Wt % of UV Curable Materials | Number of strokes req'd |
|---|---|---|
| 1 | 19% | >70 |
| 2 | 15% | 32 |
| 3 | 11% | 27 |
| 4 | 8% | 20 |

The improvement in wear performance when the amount of UV curable materials was at least 19% by weight of the composition was unexpected and surprising. As there is almost no increase in removal resistance from 11% to 15%, the large increase in removal resistance from 15% to 19% is a very surprising benefit.

Further, additional testing has shown the formulations can provide 2 weeks of wear, with high shine performance and chip resistance. Additionally, when removing the nail polish, the time the nails need to be soaked in acetone is greatly reduced as compared to conventional UV gel formulations—from 10 minutes or more to under 2 minutes.

Various modifications may be made to the various aspects disclosed herein, such modifications being contemplated as being within the scope of the invention. For example, while a specific order of steps or arrangement of functional elements is presented in the various embodiments described herein, various other orders/arrangements of steps or functional elements may be utilized within the context of the various embodiments. Further, while modifications to embodiments may be discussed individually, various embodiments may use multiple modifications contemporaneously or in sequence, compound modifications and the like.

Although various embodiments which incorporate the teachings of the present invention have been shown and described in detail herein, those skilled in the art can readily devise many other varied embodiments that still incorporate these teachings. Thus, while the foregoing is directed to various embodiments of the present invention, other and further embodiments of the invention may be devised without departing from the basic scope thereof. As such, the appropriate scope of the invention is to be determined according to the claims.

What is claimed is:

1. A cosmetic nail composition comprising:
an ultraviolet (UV) gel portion comprising:
a plurality of UV-curable materials, the plurality of UV-curable materials present in a total amount of at least 19% by weight of the cosmetic nail composition, the plurality of UV-curable materials including a difunctional acrylate oligomer; and
a photoinitiator; and
a solvent-based portion comprising a plurality of solvents.

2. The cosmetic nail composition according to claim 1, wherein the difunctional acrylate oligomer comprises a urethane monomer, and the plurality of UV-curable materials consists of the urethane monomer and a polymerizable (meth)acrylate.

3. The cosmetic nail composition according to claim 2, wherein the urethane monomer is a urethane acrylate monomer, and the polymerizable (meth)acrylate is pyromellitic dimethacrylate.

4. The cosmetic nail composition according to claim 3, wherein the urethane acrylate monomer is present in an amount from 8-15% by weight of the cosmetic nail composition, and the pyromellitic dimethacrylate is present in an amount from 2-8% by weight of the cosmetic nail composition.

5. The cosmetic nail composition according to claim 1, wherein the photoinitiator is a type I photoinitiator.

6. The cosmetic nail composition according to claim 5, wherein the type I photoinitiator is ethyl trimethylbenzoyl phenylphosphinate.

7. The cosmetic nail composition according to claim 1, wherein the UV gel portion is present in a total amount of 19-50% by weight of the cosmetic nail composition.

8. The cosmetic nail composition according to claim 7, wherein the photoinitiator is present in a total amount of 3-6% by weight of the cosmetic nail composition.

9. The cosmetic nail composition according to claim 1, wherein the solvent-based portion further comprises at least one film forming agent.

10. The cosmetic nail composition according to claim 9, wherein the at least one film forming agent comprises a cellulose film forming agent.

11. The cosmetic nail composition according to claim 10, wherein the cellulose film forming agent is nitrocellulose and/or cellulose acetate butyrate.

12. The cosmetic nail composition according to claim 10, wherein the at least one film forming agent comprises an acrylate film forming agent.

13. The cosmetic nail composition according to claim 12, wherein the acrylate film forming agent includes acrylates copolymer, acrylates/isobornyl acrylate copolymer, and/or tricyclodecanedimenahnol diacrylate.

14. The cosmetic nail composition according to claim 12, wherein the at least one film forming agent comprises one or more co-film forming agents.

15. The cosmetic nail composition according to claim 14, wherein the one or more co-film forming agents includes adipic acid/neopentyl glycol/trimellitic anhydride copolymer and/or hydrogenated acetophenone/oxymethylene copolymer.

16. The cosmetic nail composition according to claim 15, wherein the one or more co-film forming agents comprises an epoxy resin.

17. The cosmetic nail composition according to claim 16, wherein the epoxy resin is a tosylamide epoxy resin.

18. The cosmetic nail composition according to claim 14, wherein the acrylate film forming agent is present in a total amount, by weight, that is less than both the total amount of co-film forming agent and the total amount of cellulose film forming agent.

19. The cosmetic nail composition according to claim 14, further comprising a colorant.

20. The cosmetic nail composition according to claim 19, further comprising at least one additional component, the at least one additional component being present in a total amount of no more than 5% by weight of the cosmetic nail composition.

21. The cosmetic nail composition according to claim 19, further comprising a filler, a pH adjusting agent, a UV absorbing agent, or a combination thereof.

22. The cosmetic nail composition according to claim 1,
wherein the plurality of UV-curable materials comprises a first UV-curable material and second UV-curable material, the first UV-curable material present in an amount of 8-15% by weight of the cosmetic nail composition, the second UV-curable material present in an amount of 2-8% by weight of the cosmetic nail composition;
wherein the photoinitiator is present in an amount of 2-8% by weight of the cosmetic nail composition;
wherein the plurality of solvents is present in a total amount of 50-70% by weight of the cosmetic nail composition; and
wherein the cosmetic nail composition further comprises:
a plurality of film formers present in a total amount of 5-20% by weight of the cosmetic nail composition;
a UV absorbing agent present in a total amount of no more than 1% by weight of the cosmetic nail composition;
a pH adjusting agent present in a total amount of no more than 1% by weight of the cosmetic nail composition;
a filler present in a total amount of no more than 2% by weight of the cosmetic nail composition; and
a colorant;
wherein the cosmetic nail composition is substantially free of all other materials.

23. A method for providing a nail composition with improved wear, comprising:
applying a composition according to claim 1 to a nail; and
curing the composition by exposing the composition to an ultraviolet (UV) light.

24. The method according to claim 23, further comprising removing the composition after a period of time.
